(19) United States
(12) Patent Application Publication
(10) Pub. No.: US 2018/0111010 A1
(43) Pub. Date: Apr. 26, 2018

(54) Method and Apparatus for Facilitating Pain Relief

(71) Applicant: Pohling Kuo, Seguin, TX (US)

(72) Inventor: Pohling Kuo, Seguin, TX (US)

(21) Appl. No.: 15/788,655

(22) Filed: Oct. 19, 2017

Related U.S. Application Data
(60) Provisional application No. 62/410,413, filed on Oct. 20, 2016.

(51) Int. Cl. A61N 7/02

(52) U.S. Cl. CPC A61N 7/02

(57) ABSTRACT

A method and apparatus for facilitating pain relief utilizes ultrasound waves to generate heat in a body acupuncture meridian to relieve pain. The pain relief apparatus has a housing containing a transducer element, which transmits ultrasonic waves through one or more transmitting heads to a user's skin. A massaging portion is furthermore connected to the housing. The method allows a user to choose one or more body acupuncture meridians to treat with the pain relief device using a smartphone application, which then displays instructions to the user on how to effectively apply treatment. The smartphone application may furthermore be capable of controlling the functionality of the pain relief device.

The current application claims a priority to the U.S. Provisional Patent application Ser. No. 62/410,413 filed on Oct. 20, 2016.

### FIELD OF THE INVENTION

The present invention relates generally to pain relief. More particularly, the present invention relates to a method and apparatus for facilitating pain relief through application of ultrasound waves to a portion of an individual's body acupuncture meridian.

### BACKGROUND OF THE INVENTION

Pain management has become a large market, in large part today due to pharmaceuticals. Although drugs may relieve pain for individuals, some of the drugs may leave an individual with more problems than before the individual had taken the drugs. Problems may include negative side effects and addiction.

The diagnosis of a disease may be a very different process when comparing diagnoses in Eastern and Western medicine. Western medicine may attempt to locate an external cause to explain a disease while Eastern medicine may attempt to analyze internal energy forces and where this energy is being blocked within the body (causing disease). In eastern medicine, and specifically acupuncture practice, the body is recognized as containing 14 meridians/energy channels: three on the front arm, three on the posterior arm, three on the front leg, three on the posterior leg, one on the front of the body (Yin), and one on the posterior of the body (Yang). These specific points may be manipulated in order to provide pain relief to specific parts of the body. Typically, acupuncture and similar holistic approaches to relieve pain require attention to the ailing part of the body, which may not work or may take a substantial amount of time before the individual feels better.

Western Medicine believes that the human body is basically a sophisticated machine made up of distinct parts and this has led to many extraordinary advances in technology, science, and treatments. However, this approach to health and healing fails to understand the extremely powerful interactions between the body, mind and spirit and the concept that human beings are more than the sum of their parts.

The Chinese Medicine believes that health is impacted by Yin and Yang forces, and that disease is caused by disruptions and imbalances in the flow of qi (vital life energy) through the body. The techniques attempt to unblock congested energy pathways (meridians), thus allowing a freely flowing energy current (qi) throughout the body. Life considered as a bio-electrical/vibrational energy phenomenon.

Yin and Yang are general term used to describe interdependent aspects of objects or natural phenomena such as male and female, heaven and earth, day and night, and water and fire.

The natures of Yin and Yang are relative. They always exist together as variable proportions of each other just as the concept of hot has no meaning without cold, and day has no meaning without night. Yin and Yang describe a dynamic interaction between all aspects of nature and the universe. They express the idea of opposing but complementary phenomena that exist in a state of dynamic equilibrium. Yin represents cold, earth, moon, night, autumn, female, rest, downwardness, and passivity. Yang represents warmth, light, heaven, sun, summer, male, fast, excitement, upwardness, and vigor.

Western Medicine tends to view the human being as a group of specialized organs and functions. Traditional Chinese Medicine is more concerned with the dynamics of interrelationships, the patterns of vital energy in the person.

Qi is the basic vital substance that circulates with strong energy. A “life force,” “vital force,” “matter-energy,” and “breath.” Eastern Medicine study and observe the flow, rhythm, cycles, changes, movement, and balance of Qi. Qi is impulsive, it warms the body, protects the body, transforms food and air into the vital substance of blood, and it controls the body by holding organs and vessels. Current attempts to combine acupuncture and western medical technology focus on applying an electrical stimulation to an acupuncture pressure point on the body, thereby creating an electrical circuit with the body.

It is an objective of the present invention to present a method and apparatus for relieving pain in an individual's body through application of heat generated through ultrasound waves.

### BRIEF DESCRIPTION OF THE DRAWINGS

FIG. 1 is a front diagrammatic view of a portion of the 12 meridians of a human body in accordance with some embodiments.

FIG. 2 is a rear diagrammatic view of a portion of the 12 meridians of a human body in accordance with some embodiments.

FIG. 3 is a perspective view of the pain relieving apparatus with dual transmitting heads in accordance with some embodiments.

FIG. 4 is a general schematic diagram of electronic components of the pain relieving apparatus in accordance with some embodiments.

FIG. 5 is a perspective view of the pain relieving apparatus with a single transmitting head in accordance with some embodiments.

FIG. 6 is an illustration of the pain relieving apparatus being applied to a user for pain relief in accordance with some embodiments.

FIG. 7 is a stepwise flow diagram of the general steps of the method of the present invention in accordance with some embodiments.

FIG. 8 is a stepwise flow diagram showing steps for displaying the body part menu in the method of the present invention in accordance with some embodiments.

FIG. 9 is a stepwise flow diagram showing steps for activating the pain relieving device in the method of the present invention in accordance with some embodiments.

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced or utilized without the implementation of some features as they are described. It should be understood that some details have not been described in detail in order to not unnecessarily obscure focus of the invention. References herein to “the preferred embodiment”, “one embodiment”, “some embodiments”, or “alternative embodiments” should be considered to be illustrating aspects of the present invention that may potentially vary in some instances, and should not be considered to be limiting to the scope of the present invention as a whole.

It will be understood that, although the terms first, second, third, etc. may be used herein to describe various elements, these elements should not be limited by these terms. These terms are only used to distinguish one element from another element. Thus, a first element discussed below could be termed a second element without departing from the teachings of the present disclosure. The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprises” and/or “comprising” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

The present invention presents a technique intended to combine ancient practices for stimulating the Qi (energy channel) of the body according to Eastern medicine with Western medicine modality, ultrasound, to restore balance and harmony to the individual. The therapy treatment is used to relieve pain and to increase energy and wellness.

The present invention generally provides an apparatus for facilitating pain relief through application of ultrasound waves, and an accompanying method.

The apparatus for facilitating pain relief in the present invention may be understood generally to be a tool which may be manually operated by a medical professional or by a user to perform pain relief therapies on themselves in the convenience of their own home.

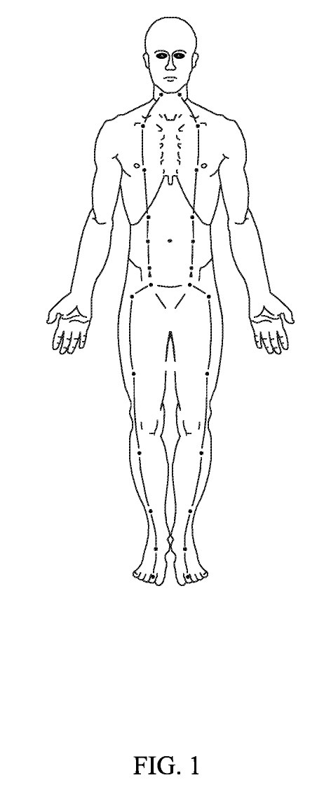

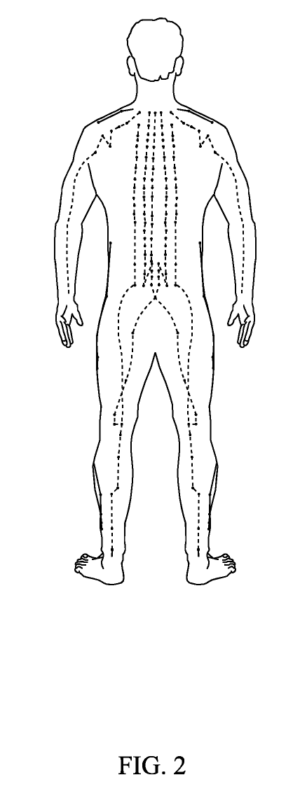

A depiction of a human body may display three technical points on each limb of the body: the left arm, the right arm, the left leg, and the right leg. The depiction may additionally include a single technical point near a central point of the body. FIG. 1 displays a rear diagrammatic view may display of a portion of the 12 meridians in accordance with some embodiments. The depiction of a body may display a single technical point near a central point of the body. FIG. 2 displays a rear diagrammatic view of a portion of the 12 meridians in accordance with some embodiments. The technical points may denote where heat may be applied to the body.

Depending on where a pain is identified within an individual's body, heat may be applied to a specific technical point. When heat is applied, the technical point is stimulated; this may allow the brain to release pain-killing and anti-inflammatory substances that may be “fed” to the ailing portion of the body. The application of heat may further allow muscles in the body to relax as well as increase blood circulation to the ailing portion of the body. In embodiments, heat may be applied to multiple technical points in order to alleviate pain from a single area on the body. By stimulating the energy channel at the technical points, the energy level of an individual may be increased.

For example, if an individual has a pain in their shoulder, heat may be applied to the technical point located on the back of the individual as well as on the forearm of the individual. The combination of heat applied to the two areas may provide at least one of pain-killing substances and anti-inflammatory substances to the shoulder and may additionally relax contracted muscles in the shoulder and/or provide increased blood circulation to the area and increase energy levels of the individual.

A thermal agent apparatus may be utilized to apply head to an individual's body in accordance with some embodiments. In the preferred embodiment, the thermal agent apparatus may be an ultrasonic apparatus. The thermal agent apparatus may provide ultrasonic waves to an individual's body in the range of 1 watt per centimeter squared to 5 watts per centimeter squared. When the ultrasonic waves are applied to the individual's body by the thermal agent apparatus, heat may additionally be delivered via the ultrasonic waves. When applied, the thermal agent apparatus may perform at least one of the following: increase bloodflow within the body, remove knots, decrease stiffness, and increase the energy level within the body.

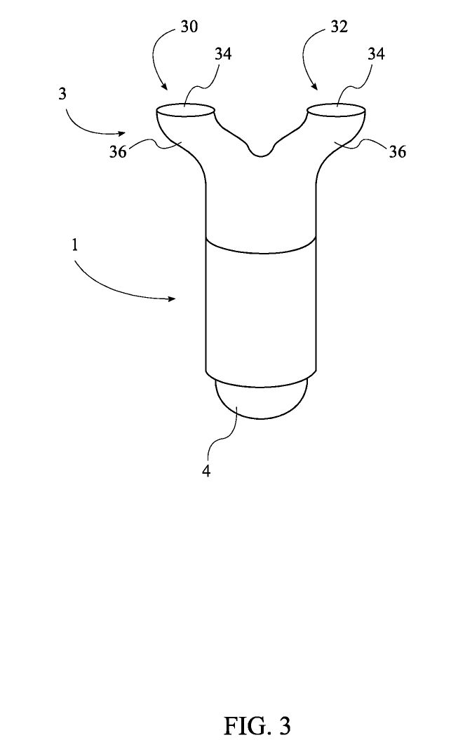

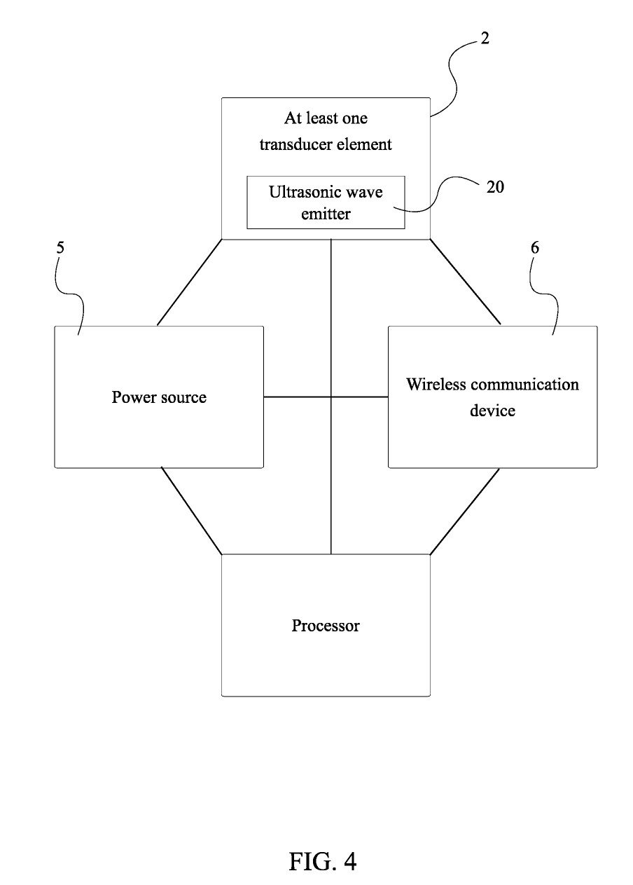

Referring to FIGS. 3-4, in some embodiments, the apparatus for facilitating pain relief of the present invention may generally comprise a housing 1, at least one transducer element 2, at least one transmitting head 3, a massaging portion 4, and a power source 5. FIG. 3 displays an illustration of one embodiment of the apparatus for facilitating pain relief, and FIG. 4 displays a general schematic diagram of the electronic components of the apparatus.

The transducer element 2 is configured to produce a pain relieving effect field. The transducer element 2 is operatively coupled to each of the at least one transmitting head 3 such that the at least one transmitting head 3 transmits the pain relieving effect field from the transducer element 2 to a user's body. Each of the at least one transmitting head 3 is terminally connected to the housing 1, and in some embodiments the massaging portion 4 is terminally connected to the housing 1 opposite the at least one transmitting head 3. In some embodiments, the massaging portion 4 may be connected elsewhere on the housing 1. Some embodiments may comprise more than one massaging portion 4. In some embodiments, the massaging portion 4 is rounded, spherical, or ball-shaped. In some embodiments, the massaging portion 4 is a half-sphere. In other embodiments, the massaging portion 4 may vary in shape and size. The massaging portion 4 may be made of a plastic or rubber material, or any other desirable material. In some embodiments, the power source 5 is a rechargeable battery that may be removable or non-removable. In some embodiments, the power source 5 is a wired connection configured to interface with a standard electrical wall outlet. The power source 5 is electrically connected to any components of the present invention that require electrical power to function. Specifically, the power source 5 is electrically connected to the transducer element 2.

In the preferred embodiment of the present invention, the transducer element 2 is an ultrasonic wave emitter 20, and thus the pain relieving effect field consists of ultrasonic waves. The transducer element 2 may be affixed to a transmitted that may transmit ultrasonic signals from the transducer element 2 to a transmitting head. The transmitting head may be applied to an individual's skin in order to apply the ultrasonic signals to the skin, which may additionally provide heat to the skin. In other embodiments, the transducer element 2 may be another type of signal or wave producing element such as, but not limited to, an electrical or magnetic field producing element.

In some embodiments, the ultrasonic wave emitter 20 is configured to produce ultrasonic waves at a rate in the range of 1 megahertz (MHz) to 15 MHz. In some embodiments, the ultrasonic wave emitter 20 is configured to produce ultrasonic waves at a rate in the range of 1 MHz to 5 MHz. In some embodiments, the ultrasonic wave emitter 20 is configured to produce ultrasonic waves at a rate in the range of 2 MHz to 15 MHz. Furthermore, in some embodiments the ultrasonic wave emitter 20 is configured to produce ultrasonic waves consisting of pulses generated at a rate in the range of 2 microseconds to 3 microseconds in duration. In some embodiments, the ultrasonic wave emitter 20 is configured to produce ultrasonic waves at a rate of power in the range of 1 watt per cubic centimeter to 5 watts per cubic centimeter. In some embodiments, the ultrasonic wave ultrasonic wave emitter 20 may be configured to produce ultrasonic waves at a rate as high as 10 watts per cubic centimeter. It is noted herein that approximately 5 watts per cubic centimeter has been observed to be consistently effective in the treatment of an individual's bodily pain. In some embodiments, the ultrasonic wave emitted may be manually configured through a user interface such as a dial, control panel, wireless connection, or other means to emit ultrasonic waves at a custom frequency, pulse duration, and power delivery.

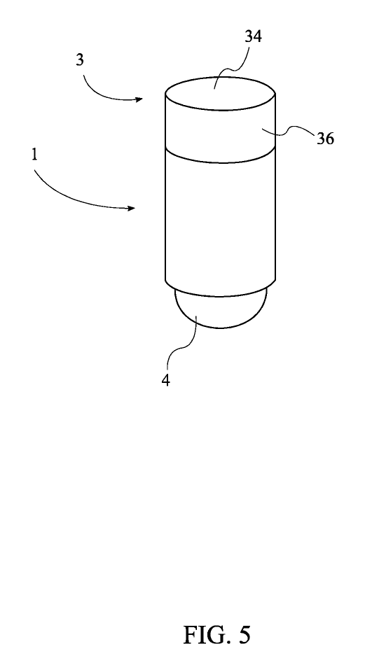

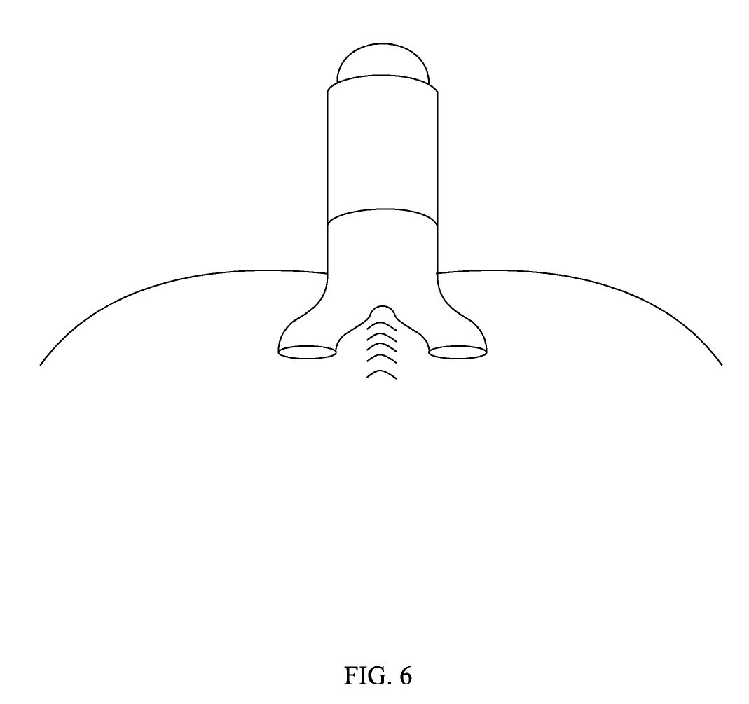

In some embodiments, the at least one transmitting head 3 comprises a single transmitting head, as shown in FIG. 5. In some embodiments, in order to properly stimulate multiple technical points or a larger area surrounding a technical point, the apparatus may include a dual head portion; thus, the at least one transmitting head 3 may comprise a first transmitting head 30 and a second transmitting head 32, as shown in FIGS. 1 and 6. In such embodiments, the first transmitting head 30 and the second transmitting head 32 are positioned adjacent to each other. The transducer element 2 may be wired to both the first transmitting head 30 and the second transmitting head 32 and thus provide ultrasonic signals to both transmitting heads. Dual transmitting head embodiments allow the transmitting heads to be positioned in two specific areas of the patient's body simultaneously.

In some embodiments, each of the at least one transmitting head 3 comprises a transmitting surface 34 and a neck portion 36. The neck portion 36 is connected to the housing 1, and the transmitting surface 34 is positioned adjacent to the next portion opposite the housing 1. Thus, the transmitting surface 34 is separated from the housing 1 by the neck portion 36. In some embodiments, an affixing point between the neck portion 36 and the transmitting surface 34 portion of the transmitting heads may comprise a ball joint. The ball joint may allow for increased mobility of the heads so that when the heads are applied to an individual's skin, the heads may conform to the geography of the individual's skin. For example, if one of the heads is applied to a portion of an individual's back where a bone is sticking out, the head may more easily traverse the area due to the increased mobility provided by the ball joint configuration.

In some embodiments, each of the at least one transmitting head 3 may be removably attached to the housing 1. In some embodiments, the neck portions 36 of the transmitting head and/or dual head portion may be removably engageable to the housing 1 of the apparatus. In some embodiments, the transmitting head and/or dual head portion may be removably engageable via male-female engagement. The apparatus may comprise at least one receiving slot for receiving a protruding portion on a neck base of the transmitting head/dual head portion. When the neck base is placed within the apparatus, the transmitting head/dual head portion may be locked into the apparatus via actuation. In embodiments, the neck base may be pushed into the apparatus to the extent that the transmitting head/dual head portion locks in place. In some embodiments, the neck base may be twisted within the apparatus until the transmitting head/dual head portion locks in place.

In some embodiments, the apparatus for facilitating pain relief of the present invention may further comprise a wireless communication device 6. The wireless communication device 6 may facilitate electronic communication with another device for various purposes, including, but not limited to, controlling various attributes of the generated, ultrasonic waves, providing feedback to the user, and other functionalities. The wireless communication device 6 is electrically connected to the power source 5, is positioned within the housing 1, and may be communicably coupled with a user interface device. Furthermore, the present invention may comprise a processor. The processor may be understood to be any electronic integrated circuit (IC), printed circuit board (PCB), microprocessor, or combination of any electronic components that facilitates receipt of input electronic signals, processing said signals, and producing output electronic signals, in addition to any other electronic computations that may be desirable to implement in the present invention.

As illustrated in FIG. 6, a general method of use of the apparatus may comprise identifying a first portion of a body. In some embodiments, the first portion of a body may be an acupuncture point. The first portion may be proximate a pain found within an individual's body (this first portion may alternatively be known as a “complaint point”). In embodiments, conditions that induce the pain that may be relieved may include, but are not limited to: Charlie horse, sprains, edema, bursitis, scar tissues, sacro-lumbar strain, pulled muscles, pulled tendons, sciatica, neck pain, shoulder pain, arm pain, hip pain, knee pain, lower back pain, neuropathy, fibromyalgia, carpal tunnel syndrome, stress, and menstrual cramps. A heating agent, such as that previously described, may be provided. Features of the heating agent may vary depending on the type of pain felt by the individual.

The heating agent may then be contacted to at least a second portion of an individual's skin. The at least a second portion may be a predetermined vicinity from the first portion. The second portion may correlate with an energy channel found on an individual's body. The vicinity of the at least a second portion may be determined based on the complaint point. The heating agent may be applied to technical points found on the energy channel for 3-5 minutes, depending on the location of the second portion. In some embodiments, the heating agent may be applied up to three time in one day. In some embodiments, the heating agent may be applied to the second portion in a circular motion. Once the heating agent has been applied to the second portion, the heating agent may be applied to a third portion of an individual's body. This third portion may include the “Yang” portion of an individual's body, which may correlate with the spine of an individual. In some embodiments, the third portion may be stimulated for the same time period that the second portion is stimulated. In certain circumstances, pain may be relieved during treatment.

After stimulating the technical points (increasing internal energy and bloodflow), it may be recommended that the individual drink water that is close to room temperature or warmer. This may induce urination, which may allow for toxins to flush out of the body (which may also be done by sweating). It may be recommended that cold water be avoided because it may take the body internal energy in order to warm up the cold water being ingested. This energy may be necessary to be utilized in the healing process of the pained area.

In conjunction with the apparatus, the present invention furthermore presents a method of facilitating pain relief. It may be understood that the method of the present invention is primarily disclosed in relation to a method of operating an application installed on a smartphone, tablet, personal computer, or other similar electronic device capable of running such a digital application. Therefore, the method of the present invention may be understood to be facilitated by executing computer-executable instructions stored on a non-transitory computer-readable medium.

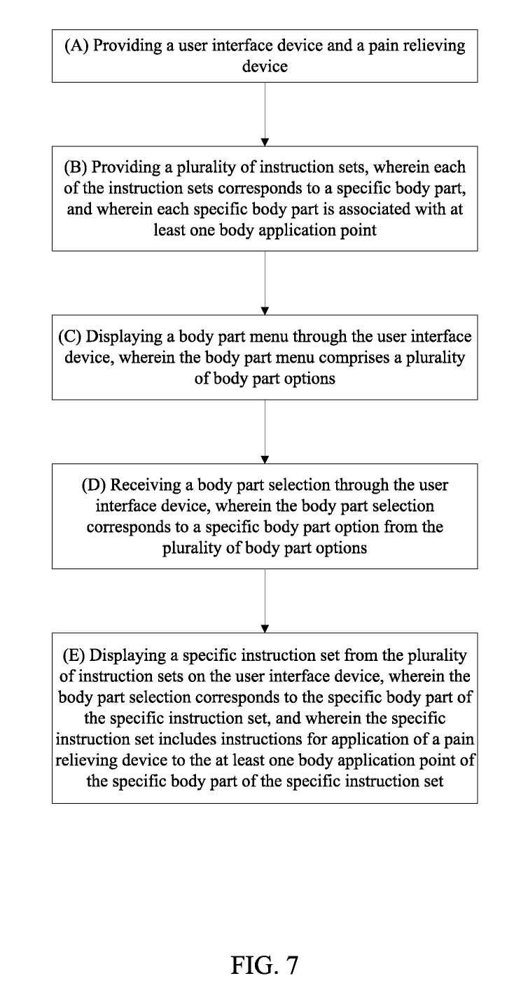

Referring to FIG. 7, in the method of the present invention, a user interface device is provided, in addition to a pain relieving device (Step A). Said pain relieving device may be understood to be the apparatus of the present invention previously herein disclosed. The user interface device may be, but is not limited to, a hand-held smartphone, tablet, personal computer, laptop computer, wall-mounted control panel, or any other electronic device capable of facilitating the steps of the method of the present invention hereinafter disclosed. The method of the present invention may be understood in general to provide a first functionality of presenting functional instructions to the user pertaining to operating the pain relieving device, and a second functionality of initializing operation of the pain relieving device and furthermore adjusting various parameters of operation of the pain relieving device.

In the method of the present invention, a plurality of instruction sets is moreover provided, wherein each of the instruction sets corresponds to a specific body part, and wherein each specific body part is associated with at least one body application point (Step B). Each specific body part may be associated with two or more body application points, or technical points.

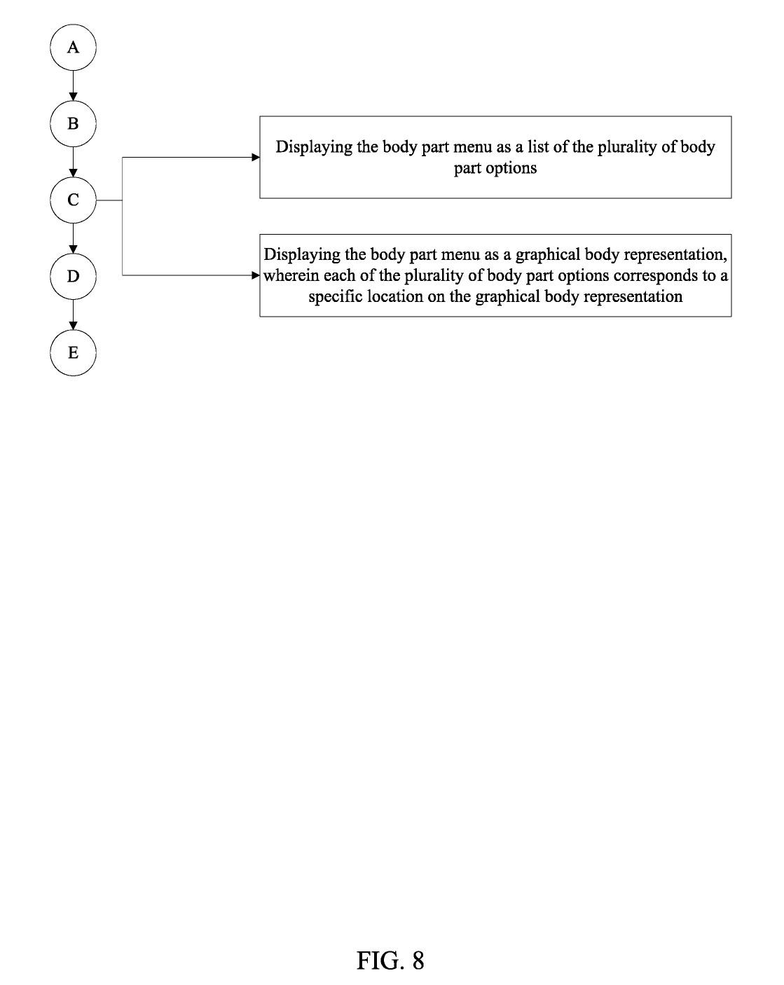

A body part menu is displayed through the user interface device, wherein the body part menu comprises a plurality of body part options (Step C). Referring to FIG. 8, in some embodiments, the body part menu is displayed as a list of the plurality of body part options. In some embodiments, the body part menu is displayed as a graphical body representation, wherein each of the plurality of body part options corresponds to a specific location on the graphical body representation. Thus, to utilize the app of the present invention, a user may select which body part they are experiencing pain in, and the app will subsequently display instructions for utilizing the pain relieving device to that purpose.

A body part selection may be received through the user interface device, wherein the body part selection corresponds to a specific body part option from the plurality of body part options (Step D).

Then, a specific instruction set is displayed from the plurality of instruction sets on the user interface device (Step E). The body part selection input by the user corresponds to the specific body part of the specific instruction set, wherein the specific instruction set includes instructions for application of a pain relieving device to the at least one body application point of the specific body part of the specific instruction set. Therefore, in the general method of the present invention, a menu is displayed to the user, from which the user selects a specific body part. Then, an instruction set is displayed to the user, providing instructions for treating the selected body part with the pain relieving device. For example, the instruction set may instruct the user to apply the pain relieving device to a first body application point for two minutes in a circular motion, and then to apply the pain relieving device to a second body application point for three minutes in a stationary position.

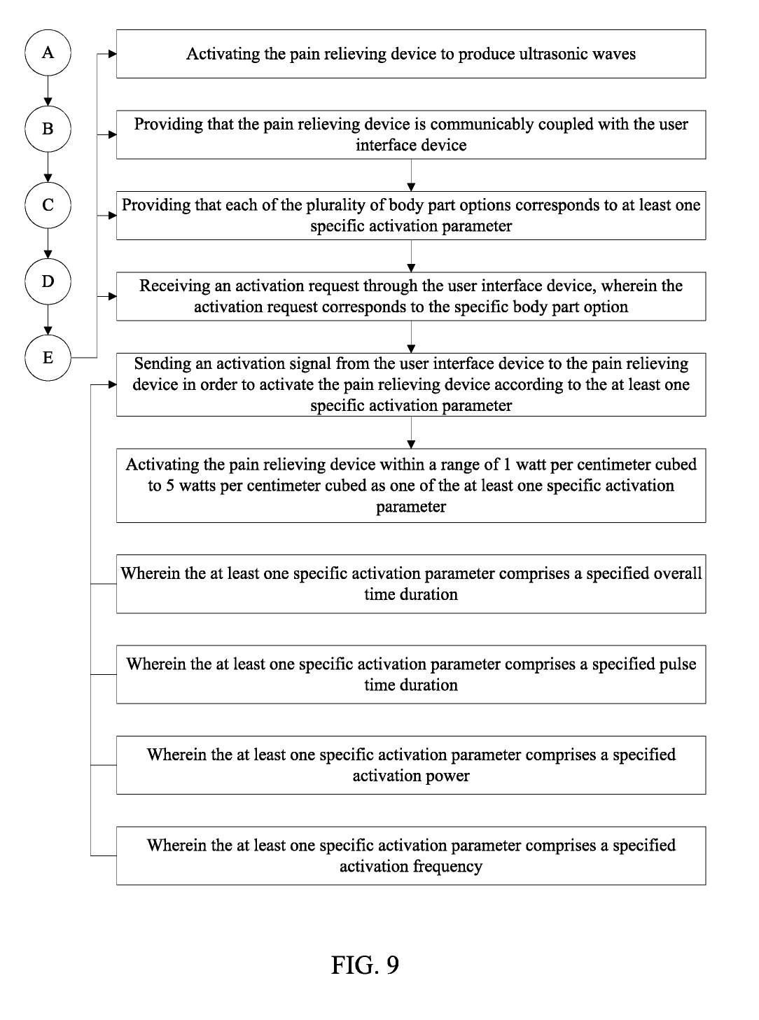

Referring to FIG. 9, in some embodiments, in addition to providing usage instructions to the user, the method of the present invention may moreover provide the ability to activate, through electronic communication using a wireless communication device, the pain relieving device to produce ultrasonic waves. Thus, providing that the pain relieving device is communicable coupled with the user interface device, and providing that each of the plurality of body part options corresponds to at least one specific activation parameter, an activation request may be received through the user interface device, wherein the activation request corresponds to the specific body part option. Then, an activation signal is sent from the user interface device to the pain relieving device in order to activate the pain relieving device according to the at least one specific activation parameter. Stated alternatively, various different body points may require different parameters for treatment. For example, a shoulder may require treatment at 5 W/cm³ for 3 minutes, whereas a thigh muscle may respond better to treatment at 7 W/cm³ for 5 minutes. Thus, in some embodiments, selecting different body parts for treatment may result in activation of the pain relieving device at different operating parameters, producing different variations of ultrasonic waves.

In some embodiments, the at least one specific activation parameter comprises a specified overall time duration. In some embodiments, the at least one specific activation parameter comprises a specified pulse time duration. In some embodiments, the at least one specific activation parameter comprises a specified activation power. In some embodiments, the at least one specific activation parameter comprises a specified activation frequency. In some embodiments, the pain relieving device is activated within a range of 1 watt per centimeter cubed to 5 watts per centimeter cubed as one of the at least one activation parameter.

Although the invention has been explained in relation to its preferred embodiment, it is to be understood that many other possible modifications and variations can be made without departing from the spirit and scope of the invention as hereinafter claimed.

What is claimed is:

1. A method of facilitating pain relief comprises the steps of:
providing a user interface device and a pain relieving device;
providing a plurality of instruction sets, wherein each of the instruction sets corresponds to a specific body part, and wherein each specific body part is associated with at least one body application point;
displaying a body part menu through the user interface device, wherein the body part menu comprises a plurality of body part options;
receiving a body part selection through the user interface device, wherein the body part selection corresponds to a specific body part option from the plurality of body part options; and
displaying a specific instruction set from the plurality of instruction sets on the user interface device, wherein the body part selection corresponds to the specific body part of the specific instruction set, and wherein the specific instruction set includes instructions for application of a pain relieving device to the at least one body application point of the specific body part of the specific instruction set.

2. The method of facilitating pain relief as claimed in claim 1 comprises the step of:
activating the pain relieving device to produce ultrasonic waves.

3. The method of facilitating pain relief as claimed in claim 1 comprises the steps of:
displaying the body part menu as a list of the plurality of body part options;

4. The method of facilitating pain relief as claimed in claim 1 comprises the steps of:
displaying the body part menu as a graphical body representation, wherein each of the plurality of body part options corresponds to a specific location on the graphical body representation.

5. The method of facilitating pain relief as claimed in claim 1 comprises the steps of:
providing that the pain relieving device is communicably coupled with the user interface device;
providing that each of the plurality of body part options corresponds to at least one specific activation parameter;
receiving an activation request through the user interface device, wherein the activation request corresponds to the specific body part option; and
sending an activation signal from the user interface device to the pain relieving device in order to activate the pain relieving device according to the at least one specific activation parameter.

6. The method of facilitating pain relief as claimed in claim 5, wherein the at least one specific activation parameter comprises a specified overall time duration.

7. The method of facilitating pain relief as claimed in claim 5, wherein the at least one specific activation parameter comprises a specified pulse time duration.

8. The method of facilitating pain relief as claimed in claim 5, wherein the at least one specific activation parameter comprises a specified activation power.

9. The method of facilitating pain relief as claimed in claim 5, wherein the at least one specific activation parameter comprises a specified activation frequency.

10. The method of facilitating pain relief as claimed in claim 5 comprises the step of:
activating the pain relieving device within a range of 1 watt per centimeter cubed to 5 watts per centimeter cubed as one of the at least one specific activation parameter.

11. An apparatus for facilitating pain relief comprises:
a housing;
a transducer element;
at least one transmitting head;
a massaging portion;
a power source;
the transducer element being configured to produce a pain relieving effect field;
the transducer element being operatively coupled to each of the at least one transmitting head, wherein the at least one transmitting head transmits the pain relieving effect field from the transducer element to a user's body;
each transmitting head being terminally connected to the housing;
the massaging portion being terminally connected to the housing opposite the at least one transmitting head;
the power source being electrically connected to the transducer element;

12. The apparatus for facilitating pain relief as claimed in claim 11 comprises:
the transducer element being an ultrasonic wave emitter.

13. The apparatus for facilitating pain relief as claimed in claim 12 comprises:
the ultrasonic wave emitter being configured to produce ultrasonic waves at a rate in the range of 1 megahertz to 15 megahertz.

14. The apparatus for facilitating pain relief as claimed in claim 12 comprises:
the ultrasonic wave emitter being configured to produce ultrasonic waves consisting of pulses generated at a rate in the range of 2 microseconds to 3 microseconds in duration.

15. The apparatus for facilitating pain relief as claimed in claim 11 comprises:
each of the at least one transmitting head comprises a transmitting surface and a neck portion;
the neck portion being connected to the housing;
the transmitting surface being positioned adjacent to the neck portion opposite the housing;

16. The apparatus for facilitating pain relief as claimed in claim 11 comprises:
each of the at least one transmitting head being removably attached to the housing.

17. The apparatus for facilitating pain relief as claimed in claim 11 comprises:
the at least one transmitting head comprises a first transmitting head and a second transmitting head; and
the first transmitting head and the second transmitting head being positioned adjacent to each other.

18. The apparatus for facilitating pain relief as claimed in claim 11 comprises:
the massaging portion being rounded in shape.

19. The apparatus for facilitating pain relief as claimed in claim 11 comprises:
a wireless communication device;
the wireless communication device being electrically connected to the power source;
the wireless communication device being positioned within the housing; and
the wireless communication device being communicably coupled with a user interface device.
